(19) United States
(12) Patent Application Publication
(10) Pub. No.: US 2024/0093414 A1
(43) Pub. Date: Mar. 21, 2024

(54) A NONWOVEN FABRIC WITH CONDUCTIVE MEMBERS AND A METHOD TO PRODUCE IT

(71) Applicant: OFERTEX INDUSTRIES (1997) LTD., Barkan (IL)

(72) Inventor: Achiad SLOTKI, Barkan (IL)

(21) Appl. No.: 18/271,546

(22) PCT Filed: Jan. 12, 2022
(86) PCT No.: PCT/IL2022/050047

§ 371 Date: Jul. 10, 2023

(30) Foreign Application Priority Data
Jan. 12, 2021 (IL) 280117

(51) Int. Cl. D04H 1/4382; G01B 7/16; G01L 1/20; G01N 27/04

(52) U.S. Cl. CPC D04H 1/43835; G01B 7/18; G01L 1/20; G01N 27/048; D10B 2401/18

(57) ABSTRACT

A nonwoven fabric comprising at least one conductive member, wherein said at least one conductive member is embedded in said nonwoven fabric at one of the stages of the fabric production and wherein said at least one conductive member comprising a conductive material.

### FIELD OF INVENTION

The present invention is in the field of conductive fibers and threads. More particularly, the present invention is in the field of conductive fibers and threads combined in textiles and even more specifically, conductive fibers and threads embedded in nonwoven fabrics.

### BACKGROUND OF THE INVENTION

Conductive textiles have been found useful in numerous applications. Conductive embedding textile usually involves materials with different levels of conductivity. Some are substantially less than others. The conductivity level of a metal, such as copper, is much higher than that of a conductive polymer, etc. which is still sufficient to carry out various current conductivity tasks such as humidity detection.

Most conductive embedded fabrics are woven, knitted, or otherwise involve woven threads.

Most applications of conductive embedded textiles are humidity detection or warming when the embedded conductive member is either an electrode or a heating element.

When the conductive fabric is a nonwoven fabric the conductive member is sewed or glued to the material in a post-production stage. Some of the examples or previous publication of the existing conductive nonwoven fabric are enlisted herein;

US2007054577 relates to an electroconductive textile material and method of preparation thereof. The method consists mainly of two stages: 1) special pretreatment of the fabric substrate for activation and making it suitable for subsequent application and strong attachment of a conductive coating with the use of a layer-by-layer technique (LBL); 2) subsequent application and strong attachment of a conductive coating by means of a layer-by-layer technique. The first stage may be carried out thermally, thermochemically, by treating in hot solutions, or plasma-chemically by plasma treatment.

TW 201739432A relates to a nonwoven fabric with sensing moisture function, which enables the nonwoven fabric of chemical fiber to have the function of sensing moisture; the main function is: sensing the moisture nonwoven fabric containing a plurality of surface sputtered metal film, the metal film is sputtered It senses the surface of the wet nonwoven chemical fiber and the gap between the chemical fibers, so it can be extremely thin and close to the surface of the sensing moisture nonwoven fabric, achieving the effect of good conductivity and power failure, and sensing of the moisture function.

US2018279416A discloses a fabric heating element including an electrically conductive, nonwoven fiber layer having a plurality of conductive fibers collectively having an average length of less than 12 mm. The fabric heating element also including at least two conductive strips electrically connected to the fiber layer over a predetermined length, positioned adjacent opposite ends of the fiber layer, and configured to be electrically connected to a power source.

It can be clearly seen in the cited publications and many others that there is no prior art relating to a conductive embedded nonwoven fabric that is easy to manufacture and could be use to a vast variety of application.

### SUMMARY OF THE INVENTION

One object of the present invention is to provide a method to produce a nonwoven fabric with at least one conductive member embedded in it in the manufacturing process.

Another object of the present invention is to provide a conductive member embedded into a nonwoven fabric that can carry various tasks.

Yet another object of the present invention is to provide for an easy to make, easy to use nonwoven fabric with at least one conductive member.

The conductive member of the present invention could be made of any conductive material and in various forms. It could be a thread, a woven thread, a yarn, or just fibers.

The invention relates specifically to the methods of stitch-bonded and needle punched. Still, those who are skilled in the art will understand that thermal bonding, hydroentangling or chemical bonding are also suitable for implementing the technic of the invention.

The method involves introducing the conductive member into the fabric in the stage of web forming or web bonding.

The web forming stage is the arrangement of the fibers in the desired consistency and patterns to create the nonwoven fabric, and the web bonding stage is the fix of the fibers to form a stable fabric avoiding the fibers from getting lose.

The said at least one conductive member could be used in various ways to exploit its conductivity and might comprise several different materials with different conductivity thereof.

### DETAILED DESCRIPTION

The conductive member, according to the present invention, could be a thread yarn or nonwoven fibers. It could be made of conductive material or be coated with such.

The conductive member could be introduced to the nonwoven in the web formation stage and put as part of the fabric's own fibers. It could be on top of the fibers and exposed to one side. It could be on an entire area in-between areas of fibers and be exposed to both sides. Alternatively, it could be arranged in between the fabrics' fibers in a way it is covered from all sides.

The conductive material could be introduced to the nonwoven web in the web bonding stage, especially in a stitch bond fabric type. In this bonding method, the conductive thread, yarn, or fibers could be put together with the sewing thread, and sometimes it could even replace it.

Similarly, the conductive member could be introduced to the web in the needle punching stage by placing it in the course of punching and having it punched into the primary fabrics' fibers.

As described above, the primary function of conductive embedding fabrics is humidity detection. Usually, there are two conductive members arranged on both sides of a specific area of the fabric. Both conductive members are connected to a circuit, which is closed whenever the fabric in between the conductive members is wet, exploiting the nature of water and other fluids to be conductive.

The easy to produce easy to use conductive embedding nonwoven fabrics could be used in the same manner but could also amplify and vary the applications that could be performed.

The vast advantage of a non woven fabric is that it is easily can be made with different kinds of fibers on different areas of the web, allowing for a wide range of applications. It could be more absorbent or less, thick or thin, smooth or rough, etc. And it could be divided into different resisting conductivity areas or allowing for multiple multifunctional conductive members.

In some embodiments of the present invention, the conductive members are the usual two electrodes described above.

In other embodiments, the conductive material is spread in a manner that creates a matrix on the fabric. The matrix could allow for the detection of different conductivity in different areas of the fabric. In other embodiments, the conductive material could be placed in a manner that reflects gradually changing distances allowing the measuring of humidity, pressure temperature, or other parameters.

In yet other embodiments, the conductive members could be of different materials and allow to detect, for example, different levels of humidity and wetness.

In some embodiments of the present invention, the conductive members could be a part of an electronic component. Such a component could be a capacitor, a resistor transistor, or a diode. For some of these embodiments, the conductive member material is made of different semiconductor materials and arranged in the proper arrangement to allow the functionality. Such an arrangement could create, for example, a P-N junction.

The usage of the conductive members as an electronic component or a part thereof allows creating a differentiating or adjustable conductivity component, such as a potentiometer or other.

The flexibility and wide variety of nonwoven fabrics could be used for different characters of the fabric resulting in different usage of the conductive members by exploiting different characters of such members.

When creating an electronic component other than a simple electrode, the detection of humidity or the presence of fluid could be measured and supply precise and accurate information. It could be used to differentiate different types of liquids and could be used to alert from dehydration and not only wetness. Such alert is of great importance, for instance, in the field of concrete and other construction fields that require a consistent humid or wet material for an optimal outcome.

The differentiating conductivity could be achieved and exploited not only by the location of the conductive members, selection of materials, and arrangement thereof but in the manipulation made on the fabric, other than humidity. For example, a fabric could be stretched or pressed, causing the conductive member to change its dimensions.

When creating a potentiometer or other multi conductivity component, based on different positions of the conductive member in the fabric or a detector that is built in that manner, the fabric could detect, for example, the touch of a body. It could be used for detecting a load on a seat or loading shelf. This could be further used for alerting the presence of a child in a car seat, preventing the parent from forgetting her in the car. It could alert a passenger to put on a safety belt. It could also be used to determine an overweight load on a specific surface.

Such implantation could be even more expansive when it is used for pressing or touching detection on a matrix set on the fabric, as described above. In those embodiments, the fabric could function in a manner that resembles that of a touch screen by detecting different events and different places on the surface of the fabric.

### A SHORT DESCRIPTION OF THE DRAWINGS

It will be understood to those skilled in the art that the fabric of the present invention is capable of sensing humidity, but it could do much more as explained herein. The conductive members, according to the present invention, could be manipulated in a wide range of ways causing them to change dimensions, shapes, and conductivity in a measurable manner or a comparable manner. Therefore the fabric of the present invention could be used for Sensing and measuring, mechanical force, temperature, weight, and many more parameters, in a wide range of configurations, different materials, and combination of materials, as well as combining the conductive members in electronic components allowing for endless applications.

The drawings as will be described reflect some examples of the embodiment of the present invention as described.

FIG. 1 depicts different examples of the conductive member. 110 is a string made wholly or partially of a conductive material. 120 is a woven thread maid wholly or partially of a conductive material. 130 is a thread consisting of loose unwoven fibers that could be put in an area of the loose fibers of the fabric in different patterns and positions and perform the role of the conductive member. 140 is a multi-layer string that could have two or more layers, some of them or all of them of a conductive material. The layers could be of different conductivity.

FIG. 2 shows different positioning of the conductive member embedded in the nonwoven fabric 200. 102 is a conductive member located along the fabric and through its depth from the bottom surface to the top. 104 is located in the vicinity of the fabric's surface 200, and 106 is located in the fabric wherein on top and bottom it is covered by plain fibers.

FIG. 2A is a section of the fabric 200 showing the conductive member 106 between the top and bottom surfaces.

FIG. 3 is a stitch bond nonwoven fabric 200, and the conductive member 108 is one of the threads used for stitch bond the fabric. Similarly, a needle punch fabric could be made using a thread or loose conducting fibers to be located at the points of punching the fibers.

FIG. 3A is a cross-section of the fabric 200 of FIG. 3.

FIG. 4 depicts a plurality of conductive members 100 arranged on a fabric 200 as a matrix, or in a predetermined pattern and distances.

FIG. 5 depicts a conductive member 108 that contains different areas made with different conductivity. This could be achieved by using different conducting materials, combining different doses of conducting material into the thread 108, or producing a different size for each section.

FIG. 5A shows the fabric 200 with a differentiated conductive member that could be used as part of an electronic component such as semiconductor, transistor, potentiometer, capacitor, resistor, or other.

FIG. 6 demonstrates how the conductive member 100 could sense touching or other force applied to the fabric 200. The sensing or measuring is done by way of current changes reflecting the change in shape or size of the conductive member 100a. The force could stretch or compress the conductive member. Otherwise, such changes in shape and size could reflect sensitivity to temperature, humidity, or other.

FIG. 7 demonstrates how the touch or press sensing fabric could be used for a chair. In the same manner, it could be used for covering doors, walls, baggage, or other object and sense or measure force (weight), temperature, humidity, and more.

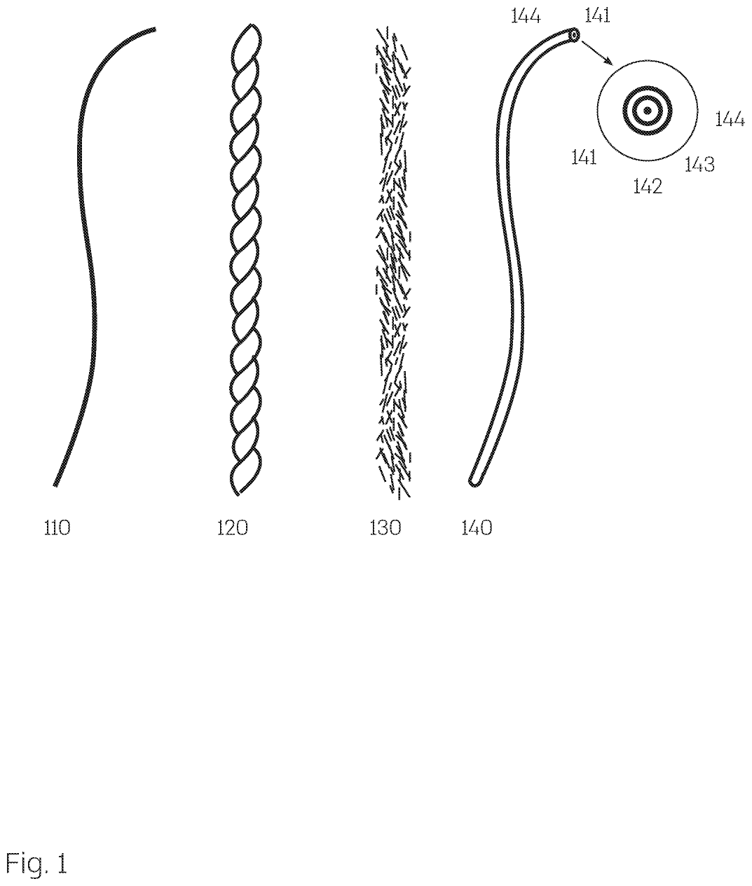

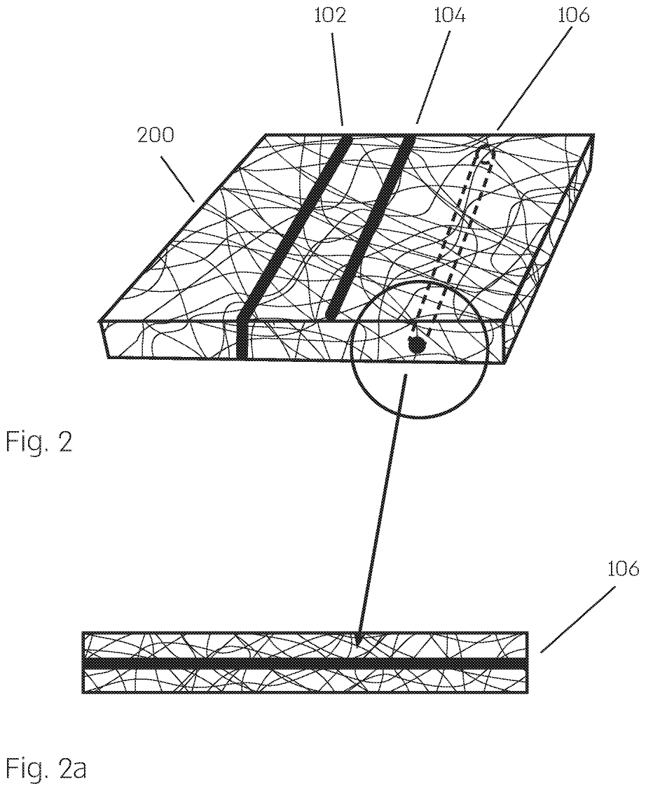

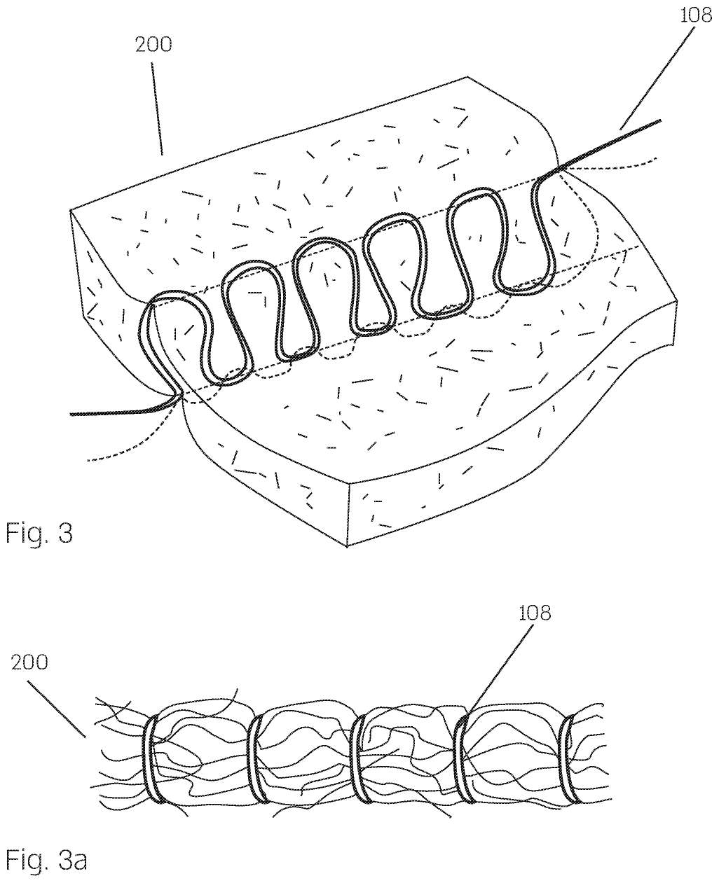

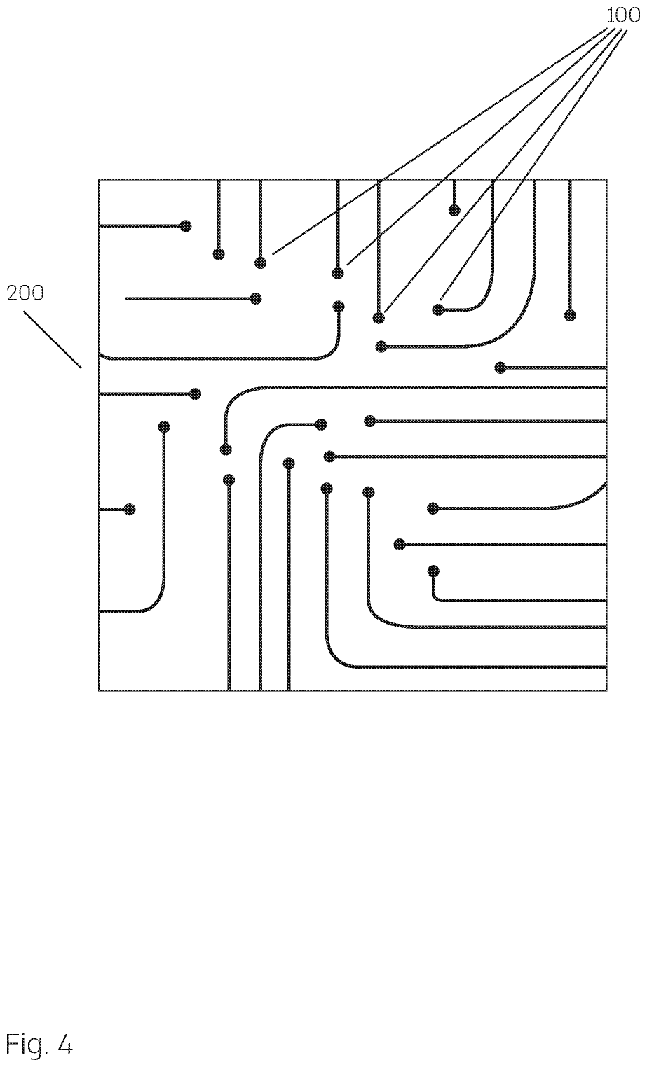

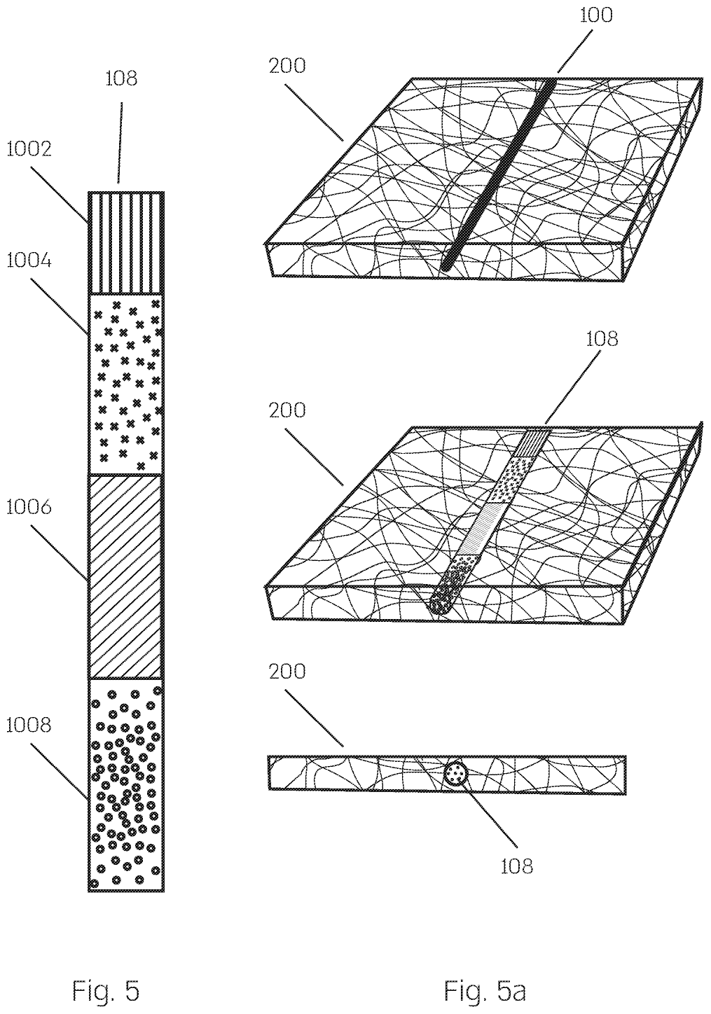

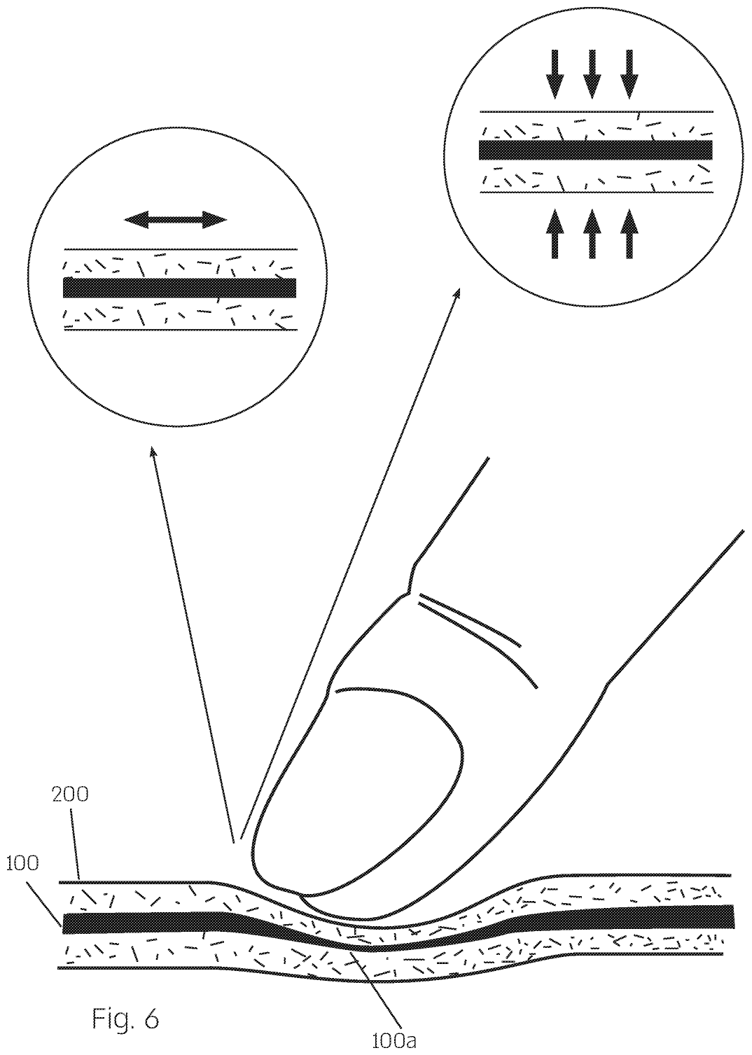

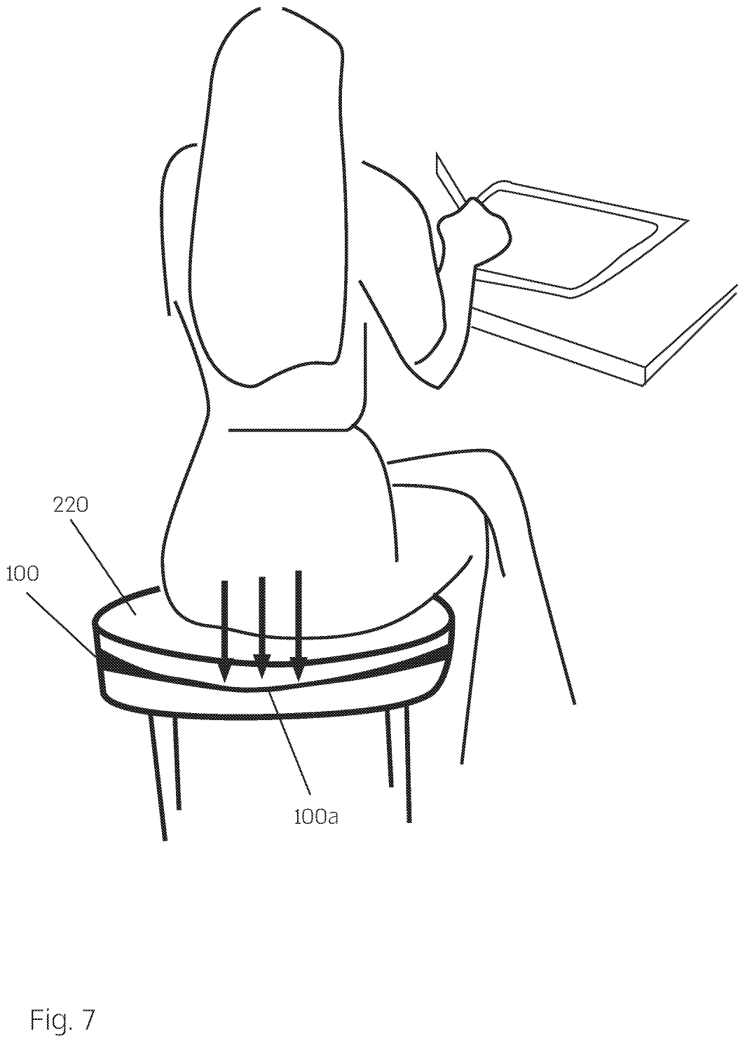

1. A nonwoven fabric comprising at least one conductive member, wherein said at least one conductive member is embedded in said nonwoven fabric at one of the stages of the fabric production and wherein said at least one conductive member comprising a conductive material.

2. A nonwoven fabric of claim 1, wherein said stage of the production, is selected from web forming or web bonding.

3. A nonwoven fabric of claim 1, wherein the bonding method is selected from stitch bond, thermal bond, needle punch, chemical bond, hydroentangling, or any combination thereof.

4. A nonwoven fabric of claim 1, wherein said at least one conductive member comprising a thread, a yarn, lose fibers, or any combination thereof.

5. A nonwoven fabric of claim 1, wherein said conductive member material is selected from metal, polymer, semiconductor, or any combination thereof.

6. A nonwoven fabric of claim 1, wherein multiple said at least one conductive member, comprising different materials with different conductivity.

7. A nonwoven fabric of claim 1, wherein said at least one conductive member consisting of said conductive material.

8. A nonwoven fabric of claim 1, wherein said at least one conductive member comprising a coating made of said conductive material.

9. A nonwoven fabric of claim 1, wherein said at least one conductive member is an electronic component.

10. A nonwoven fabric of claim 1, wherein said at least one conductive member is a part of an electronic component.

11. A nonwoven fabric of claim 9, wherein said electronic component is based on the differentiation of conductivity between multiple of said at last one conductive member.

12. A nonwoven fabric of claim 9, wherein said electronic component is based on the differentiation of conductivity within said at last one conductive member.

13. A nonwoven fabric of claim 9, wherein said electronic component is a resistor, capacitor, diode, potentiometer, transistor, or any combination thereof.

14. A nonwoven fabric of claim 1 wherein said at least one conductive member is placed on one side of the fabric on top of other fibers.

15. A nonwoven fabric of claim 1 wherein said at least one conductive member is placed on both sides of the fabric.

16. A nonwoven fabric of claim 1 wherein said at least one conductive member is placed as part of the fabric web replacing the fibers of the fabric.

17. A nonwoven fabric of claim 1 wherein said at least one conductive member is placed within the fabric in a way it is covered from all sides by the fibers of the fabric.

18. A nonwoven fabric of claim 1 wherein multiple of said at least one conductive member are placed in different areas of said fabric.

19. A nonwoven fabric of claim 18, wherein said different areas create a pattern or a matrix.

20. A nonwoven fabric of claim 1 wherein said at least one conductive member is designed to allow the detection of liquid.

21. A nonwoven fabric of claim 1 wherein said at least one conductive member is designed to allow the measurement of the level of humidity or the amount of liquid.

22. A nonwoven fabric of claim 1 wherein said at least one conductive member is designed to allow the sensing of touch of the fabric or a part thereof.

23. A nonwoven fabric of claim 1, wherein said at least one conductive member is designed to allow the measurement of force applied by touching the fabric or any part thereof.

24. A nonwoven fabric of claim 1, wherein said at least one conductive member is designed to allow the measurement of change in the shape or dimensions of said at least one conductive member, caused by mechanical force, temperature, humidity, or any combination thereof.

25. A method to produce a nonwoven fabric of claim 1 comprising three stages; web formation, web bonding, and finishing, wherein at least one conductive member embedded into the fabric and wherein said at least one conductive member embedded during said web formation or web bonding stages.
